(19) United States
(12) Patent Application Publication
(10) Pub. No.: US 2015/0238736 A1
(43) Pub. Date: Aug. 27, 2015

(54) SYSTEMS AND METHODS FOR DELIVERING DRUGS TO A TREATMENT SITE

(71) Applicant: BOSTON SCIENTIFIC SCIMED, INC., Maple Grove, MN (US)

(72) Inventors: Hongxia Zeng, Maple Grove, MN (US); Yen-Lane Chen, New Brighton, MN (US); Steven L. Kangas, Woodbury, MN (US)

(73) Assignee: BOSTON SCIENTIFIC SCIMED, INC., Maple Grove, MN (US)

(21) Appl. No.: 14/634,085

(22) Filed: Feb. 27, 2015

Related U.S. Application Data
(60) Provisional application No. 61/945,345, filed on Feb. 27, 2014.

(51) Int. Cl. A61M 25/10

(52) U.S. Cl. CPC A61M 25/1011; A61M 25/1002; A61M 2025/105; A61M 2025/1059; A61M 2025/1061; A61M 2025/1072

(57) ABSTRACT

A catheter system includes a catheter having a first balloon and a second balloon. The first balloon is longitudinally offset from the second balloon along the length of the catheter. The first and second balloons both have expanded and unexpanded states. The second balloon has a drug coating. The catheter system has a first configuration, a second configuration, and a third configuration. In the first configuration, the first balloon is in the unexpanded state and the second balloon is in the unexpanded state. In the second configuration, the first balloon is in the expanded state and the second balloon is in the unexpanded state. In the third configuration, the first balloon is in the expanded state and the second balloon is in the expanded state.

### CROSS-REFERENCE TO RELATED APPLICATIONS

This Application claims the benefit of and priority to U.S. Provisional Application No. 61/945,345, filed Feb. 27, 2014, the entire contents of which are herein incorporated by reference.

### TECHNICAL FIELD

The present disclosure pertains to medical devices, and methods for using and manufacturing medical devices. More particularly, the present disclosure pertains to drug-eluting medical devices.

### BACKGROUND

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

Expandable, implantable medical devices, such as balloons coated with drugs, can be used in a number of medical procedures for treatment of various diseases, such as coronary artery disease or peripheral artery disease. Upon inflation of the balloon, the drug is expelled from the balloon. Existing drug delivery balloons and methods, however, suffer from loss of the drug by blood flow, for example, and the amount of drug transferred to the surrounding tissue is variable. Systemic loss of therapeutic agents or drugs can decrease efficacy and present risks to a patient. In certain known systems, only a small percentage (1-10%) of the drug is actually absorbed by the surrounding tissue, thereby decreasing the drug transfer efficiency (<10% of total drug load on balloon).

Consequently, there remains a need for a drug eluting device that provides efficient drug transfer and consistent drug dosing to enhance procedural safety and efficacy.

### SUMMARY

In at least one embodiment, a catheter system has a first configuration, a second configuration, and a third configuration. The catheter system includes a catheter having a first balloon and a second balloon. The first balloon is longitudinally offset from the second balloon along the length of the catheter. The first and second balloons both have expanded and unexpanded states. In an embodiment, the second balloon has a drug coating. In the first configuration, the first balloon is in the unexpanded state and the second balloon is in the unexpanded state. In the second configuration, the first balloon is in the expanded state and the second balloon is in the unexpanded state. In the third configuration, the first balloon is in the expanded state and the second balloon is in the expanded state.

In at least one embodiment, a catheter system has a first configuration, a second configuration, and a third configuration. The catheter system includes a catheter having an inflatable balloon having a first section and a second section. The second section is longitudinally offset from the first section. The first and second sections both have expanded and unexpanded states. The second section has a drug coating and the first section is free of any drug coating. In the first configuration, the first section is in the unexpanded state and the second section is in the unexpanded state. In the second configuration, the first section is in the expanded state and the second section is in the unexpanded state. In the third configuration, the first section is in the expanded state and the second section is in the expanded state.

In at least one embodiment, a method for deploying a catheter system within a bodily lumen having a lumen wall includes providing a catheter having a first balloon and a second balloon. The first balloon is longitudinally offset from the second balloon; the second balloon has thereon a drug coating comprising a drug. The first balloon is free of any drug coating. The method further includes deploying the first balloon within the bodily lumen upstream of the second balloon such that deployment of the first balloon stagnates the fluid within the bodily lumen downstream of the first balloon. The method further comprises deploying a second balloon within the bodily lumen in the stagnated fluid, emitting the drug, and transferring the drug to the lumen wall. The drug is emitted and transferred to the lumen wall. The method additionally comprises deflating the first and second balloons and removing the catheter from the bodily lumen.

### BRIEF DESCRIPTION OF THE DRAWINGS

FIG. 1A illustrates a distal portion of an embodiment of a catheter system;

FIG. 1B is a cross-section of the distal portion of the catheter system of FIG. 1A;

FIGS. 2A-2C are schematics of an embodiment of the catheter system of FIG. 1A implanted in a body lumen, in a first configuration, a second configuration, and a third configuration, respectively;

FIG. 3A illustrates a distal portion of an embodiment of a catheter system;

FIG. 3B is a cross-section of the distal portion of the catheter system of FIG. 3A; and

FIGS. 4A-4C are schematics of an embodiment of the catheter system of FIG. 3A implanted in a body lumen, in a first configuration, a second configuration, and a third configuration, respectively;

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

References in the specification to “an embodiment”, “some embodiments”, “other embodiments”, etc., indicates that an embodiment includes a particular feature, structure, or characteristic, but every embodiment may not necessarily include the particular feature, structure, or characteristic. Moreover, such phrases do not necessarily refer to the same embodiment. Further, when a particular feature, structure, or characteristic is described in connection with an embodiment, it should be understood that such feature, structure, or characteristic may also be used in connection with other embodiments, whether or not explicitly described unless clearly evidenced or stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Some embodiments of the present disclosure are directed towards systems and methods for reducing or preventing drug particulates from being flushed away in a body lumen, e.g., reducing or preventing systemic loss. The embodiments preform this operation by temporarily reducing, restricting or stopping flow of a fluid, such as blood, within a body lumen at issue, and inflating a balloon during such reduced, restricted or stopped fluid flow. Inflation of the balloon releases the drug particulate during this reduced, restricted or stopped fluid flow and enables the drug particulate to contact the lumen wall without being flushed away. In some embodiments, a catheter system includes a catheter having a first balloon and a second balloon; the second balloon has a drug coating. The catheter system has a first configuration, a second configuration, and a third configuration. In the first configuration, the first balloon is in the unexpanded state and the second balloon is in the unexpanded state. In the second configuration, the first balloon is in the expanded state and the second balloon is in the unexpanded state. In the third configuration, the first balloon is in the expanded state and the second balloon is in the expanded state.

In some embodiments, a catheter system includes a catheter having a single balloon with multiple sections, for example, a first section and a second section; in some embodiments, the second section has a drug coating. The catheter system has a first configuration, a second configuration, and a third configuration. In the first configuration, the first section is in the unexpanded state and the second section is in the unexpanded state. In the second configuration, the first section is in the expanded state and the second section is in the unexpanded state. In the third configuration, the first section is in the expanded state and the second section is in the expanded state.

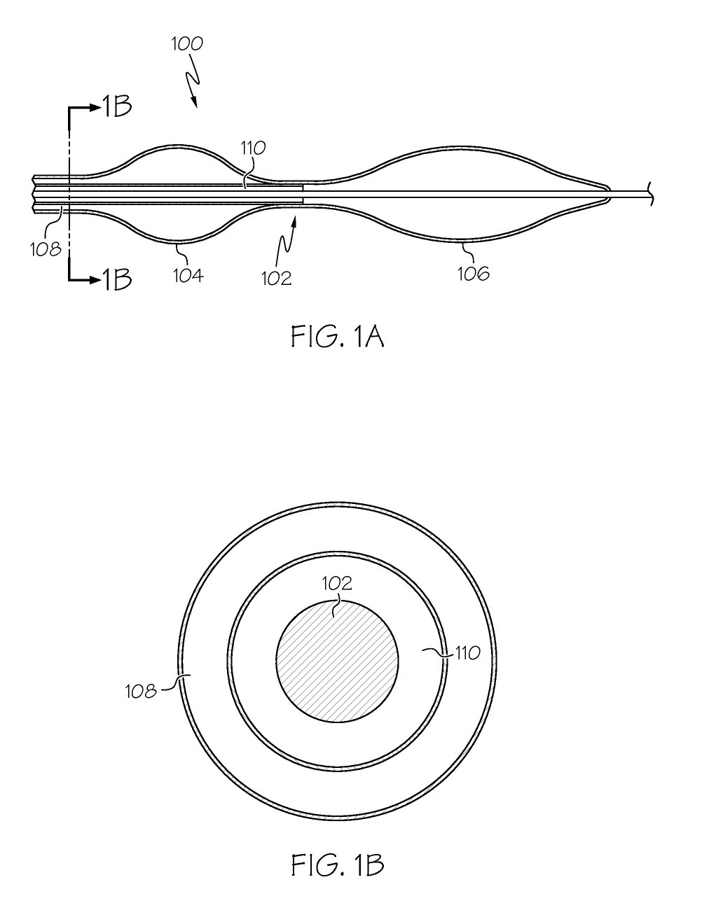

FIG. 1A illustrates a distal portion of an embodiment of a catheter system 100. In some embodiments, the distal portion of the system 100 includes a catheter 102 configured to navigate through a patient's vasculature to a desired treatment site, a first balloon 104, and a second balloon 106. The catheter 102 is an elongate shaft having a distal portion. The catheter 102 extends proximally from the distal portion to a proximal end (not shown) configured to remain outside a patient's body. The proximal end of the catheter 102 may include a hub (not shown) attached thereto for connecting other diagnostic and/or treatment devices for providing a port for facilitating other interventions. In some instances, the catheter 102 may be advanced to a desired region within the patient's vasculature using an outer sheath or guide catheter, although this may not be required.

The catheter 102 may have a long, thin, flexible tubular state. The skilled artisan will appreciate that other suitable states such as, but not limited to, rectangular, oval, irregular, or the like may also be used. In addition, the catheter 102 may have a cross-sectional shape or configuration adapted to be received in a desired body lumen. For instance, the catheter 102 may specially be sized and configured to accommodate passage through the intravascular path, which leads from a percutaneous access site in, for example, the femoral, brachial, or radial artery, to a targeted treatment site, for example, within a coronary artery.

In some embodiments, the stiffness of the catheter 102 is adapted to form a catheter system 100 for use in various body lumen diameters. To this end, the material used for manufacturing the catheter 102 includes any suitable biocompatible material such as, but not limited to, polymers, or alloys, either in combination or alone. In general, suitable polymeric materials include, but are not limited to, silicone, polyamide, polyether block amides, polyurethane, polyethylene, nylon, and polyethylene terephthalate. In some embodiments, the material employed has enough stiffness for use in various body lumen diameters, and sufficient flexibility to maneuver through tortuous and/or stenotic lumens, avoiding any undesirable tissue injuries.

In some embodiments, the catheter 102 further includes one or more lumens, such as a first inflation lumen 108 and a second inflation lumen 110, which are independent of each other. In some embodiments, each of the first and the second inflation lumens 108, 110 is proximally coupled to one or more components such as one or more fluid reservoirs (not shown), fluid regulators (not shown), and a control assembly. These components may be located adjacent to or in the hub. In some embodiments, the first and the second inflation lumens 108, 110 extend from the proximal end to the distal portion of the catheter 102. At the distal portion of the catheter system 100, in some embodiments, the first inflation lumen 108, the second inflation lumen 110, and the catheter 102 are concentric in nature (FIG. 1B). In some embodiments, the first inflation lumen 108 is located over the second inflation lumen 110, which may be adjacent to the catheter 102. Further, the first and second inflation lumens 108, 110 are in fluid communication with the balloons 104, 106, respectively and convey an inflation fluid, such as saline solution, for inflating these balloons 104, 106. The first and the second inflation balloons 104, 106 may have a variety of states and/or arrangements.

In some embodiments, the first inflation lumen 108 and the second inflation lumen 110 extend from the proximal end of the catheter 102 to the first balloon 104 and the second balloon 106, respectively.

In some embodiments, only a single inflation lumen is utilized. In some embodiments of a single inflation lumen it is proximally coupled to a single fluid regulator (not shown), one or more fluid reservoirs (not shown), and a dedicated control assembly located in the hub. Further, in some embodiments, the single inflation lumen is configured to inflate both the first and second inflation balloons 104, 106, in series. In particular, the first balloon 104 is inflated to its expanded state, via the single inflation lumen, at a first pressure. At this first pressure, however, the second balloon 106 remains unexpanded even though it is in fluid communication with the single inflation lumen and first balloon 104. After the first balloon 104 has been expanded, the second balloon 106 expands to its expanded state, via the single inflation lumen.

Thus, in some embodiments, the first and second balloons 104, 106 have different expansion characteristics (e.g., different expansion pressures). This can be achieved by selecting different materials for the first and second balloons 104, 106, for example, or using a restraining band or material on the second balloon 106, for example.

In some embodiments, one or both of the balloons 104, 106 are coaxially positioned around the catheter shaft of the catheter 102. In some embodiments, one or both of the balloons 104, 106 define the respective inflation lumens 108, 110 between an inner wall of the balloon and the outer surface of the catheter 102.

In one embodiment, the first balloon 104 is configured to restrict or stop the flow of a fluid such as blood to a treatment region; the second balloon 106 is configured to release a drug to the treatment region. The first balloon 104 may be located on either side of the second balloon 106 based on the direction of blood flow. In some embodiments, the first balloon 104 is located proximal to the second balloon 106 for restricting or stopping blood flowing from a proximal side to a distal side of the first balloon 104. Alternatively, in some embodiments, the first balloon 104 is located distal to the second balloon 106. In some embodiments, an expandable member such as the first balloon 104 is located on both sides of the second balloon 106. The first balloon 104 and the second balloon 106 have a predetermined spacing between them to avoid any interference between the balloons 104, 106 in their respective expanded states. In the expanded state, the first and the second balloons 104, 106 inflate radially and/or longitudinally. In at least one embodiment, the second balloon 106 is relatively longer than the first balloon 104.

In some embodiments, the outer surface of the second balloon 106 is covered with a drug coating, which may be a blend of a drug such as Paclitaxel or everolimus and an excipient, for example, acetyl tributyl citrate (ATBC). The drug coating may be a combination of any suitable drug and excipient based on the intended therapeutic effect and the region of treatment.

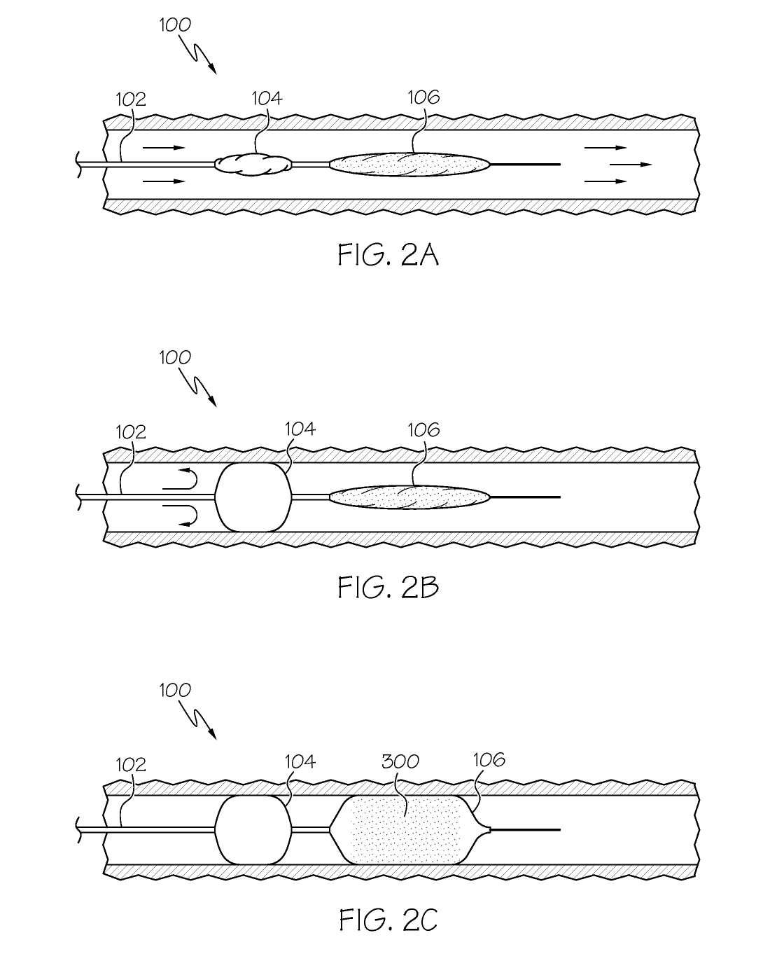

In some embodiments, as shown for example in FIGS. 2A-2C, the catheter system 100 undergoes three distinct configurations based on inflation states of the first and the second balloons 104, 106. In a first configuration (FIG. 2A), the catheter 102 is introduced within the body lumen with both the first balloon 104 and the second balloon 106 in unexpanded states; the second balloon 106 is placed adjacent to a treatment region. Once the catheter 102 has reached the treatment region, the first balloon 104 is inflated, for example by injecting an inflation fluid through the first inflation lumen 108, which is in fluid communication with the first balloon 104. The inflation fluid is injected into the first balloon 104 at a first inflation pressure until the catheter system 100 transitions to a second configuration. In the second configuration (FIG. 2B), the first balloon 104 is inflated to an expanded state while the second balloon 106 is in an unexpanded state. In the expanded state, the first balloon 104 is firmly positioned in close vicinity to the second balloon 106 such that the first balloon 104 is in contact with the lumen wall upstream of the second balloon 106 for restricting the flow of blood in the body lumen. Such deployment of the first balloon 104 stagnate the blood within the body lumen downstream of the first balloon 104.

Once the blood flow is restricted by the first balloon 104, the catheter system 100 is transitioned into a third configuration. In the third configuration (FIG. 2C), the second balloon 106 is inflated by an inflation fluid via the second inflation lumen 110, which is in fluid communication with the second balloon 106. The inflation fluid is injected into the second balloon 106 until the second balloon 106 is inflated to an expanded state; the second balloon 106 has a second inflation pressure in its expanded state. While transitioning from the unexpanded state to the expanded state, the drug coating begins to come off in the form of particulates from the outer surface of the second balloon 106 and is transferred to the surrounding tissue. Since the second balloon 106 is deployed under a static body lumen condition, wherein the blood flow adjacent to the treatment region in the body lumen is none or negligible, transfer efficiency of the drug coating particulates is improved.

In some embodiments, subsequent to the transfer of drug particulates to the treatment region, the catheter system 100 is transitioned back to the first configuration through the second configuration. In some embodiments, the second balloon 106 is deflated, followed by deflation of the first balloon 104; the catheter 102 is then removed from the body lumen.

In some embodiments, the first balloon 104 has a first inflation pressure and the second balloon 106 has a second inflation pressure. In some embodiments, the second inflation pressure in the second balloon 106 is comparable to the first inflation pressure in the first balloon 104. In some embodiments, however, the second inflation pressure in the second balloon 106 is greater that the first inflation pressure in the first balloon 104. The time lapse between consecutive deployments of the first balloon 104 and the second balloon 106 is substantially small, for example less than two seconds. Additionally, in some embodiments, the second balloon 106 has a greater volume than the first balloon 104 when the catheter system 100 is in the third configuration.

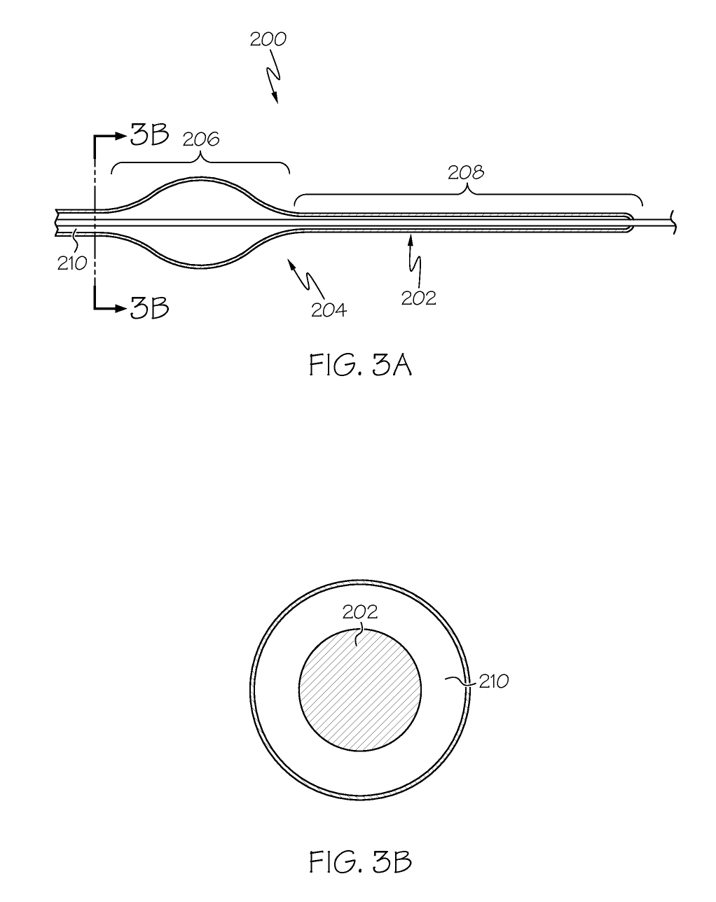

FIG. 3A illustrates an embodiment of a distal portion of a catheter system 200. The distal portion of the catheter system 200 includes a catheter 202 having an inflation lumen 210. In some embodiments, he inflation lumen 210 is proximally coupled to one or more fluid reservoirs (not shown), a single fluid regulator (not shown), and/or a dedicated control assembly (not shown), each located adjacent to or in the hub. In some embodiments, the inflation lumen 210 extends from the proximal end to the distal portion of the catheter 202. The inflation lumen 210 is concentric with the catheter 202 at the distal portion of the catheter system 200.

In some embodiments, the catheter system 200 includes a single balloon 204 having multiple sections. In at least one embodiment, the balloon 204 includes a first section 206 and a second section 208; the second section 208 has a drug coating. The single balloon 204 is disposed over the distal portion of the catheter system 200. Both the sections 206, 208 of the balloon 204 are in fluid communication with the inflation lumen 210. The first section 206 is configured to block the flow of blood and the second section 208 is configured to release a drug to a treatment region. The second section 208 is longitudinally offset from the first section 206. In some embodiments, the first section 206 and the second section 208 are separated by a region of reduced cross-section, when the single balloon 204 is fully inflated, which has a cross-sectional area smaller than that of the first section 206 or the second section 208 of the balloon 204. Additionally, in some embodiments the second section 208 is longer than the first section 206, for example to increase the area of drug delivery.

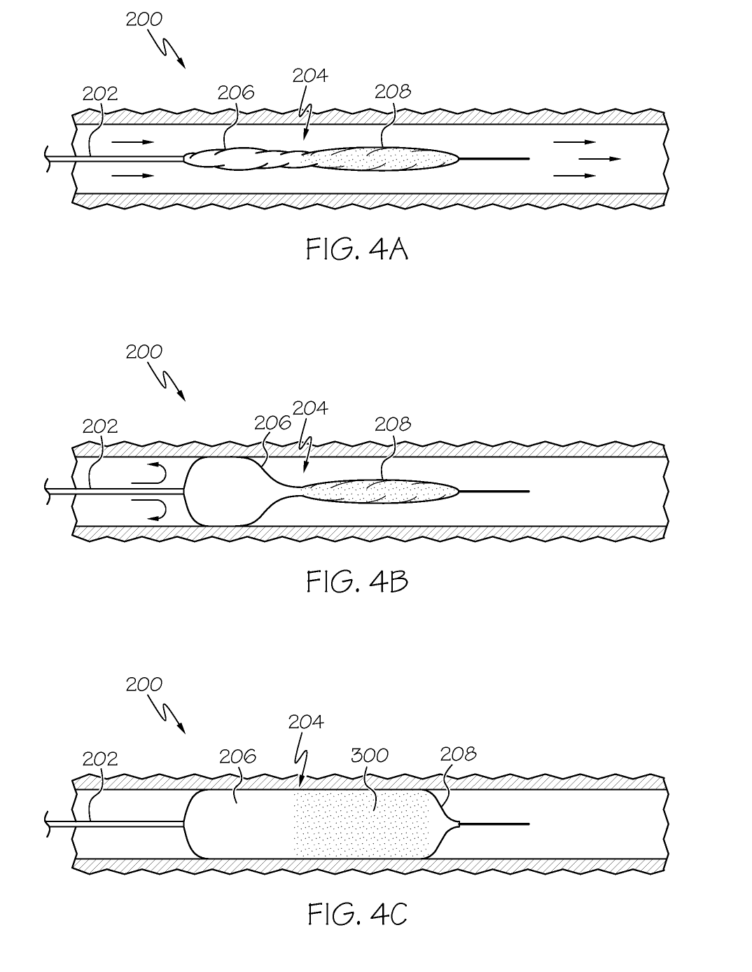

As shown in FIGS. 4A-4C, the catheter system 200 is configured to acquire three configurations based on inflation states of the first section 206 and the second section 208 of the balloon 204. During operation, the distal portion of the catheter system 200 is advanced within a body lumen and is placed adjacent to a treatment region in a first configuration. In the first configuration (FIG. 4A), the first section 206 and the second section 208 of the balloon 204 are both in unexpanded states. The balloon 204 is then injected with an inflation fluid via the inflation lumen 210 to sequentially inflate different sections of the balloon 204.

In some embodiments, the first section 206 and the second section 208 of the balloon 204 are made of different materials; for example, a material forming the second section 208 may be harder, stiffer, less elastic, or have a higher tensile modulus than that forming the first section 206 of the balloon 204. Due to such difference in the constructing material, the injection fluid inflates the first section 206 of the balloon 204 at a first inflation pressure prior to inflating the second section 208. In the second configuration, the first section 206 is inflated to an expanded state while the second section 208 is in an unexpanded state. In the expanded state, the first section 206 comes in contact with the lumen walls upstream of the second section 208, stopping the blood flow and stagnating the blood within the body lumen downstream of the first section 206.

Once the blood flow is restricted by the first section 206, the catheter system 200 is transitioned to a third configuration. In the third configuration (FIG. 4C), the inflation pressure of the inflation fluid being injected into the first section 206 is increased. As the first inflation pressure increases beyond a predetermined threshold level, pressure of the inflation fluid in the second section 208 begins to inflate the second section 208 of the balloon 204. The inflation fluid is injected into the second section 208 at a second inflation pressure until the second section 208 is inflated to an expanded state. As the second section 208 nears its expanded state, the drug coating begins to come off the outer surface of the second section 208 of the balloon 204 and is transferred to the surrounding tissue. Since the second section 208 is deployed in a momentarily stagnate environment, transfer efficiency of the drug coating is improved. Moreover, in some embodiments, the first section 206 and the second section 208 are both inflated via a single inflation lumen 210 in association with a single fluid regulator and dedicated control assembly, thereby simplifying the procedure.

Subsequent to the transfer of drug particulates 300 to the treatment region, the catheter system 200 is transitioned back to the first configuration through the second configuration. In some embodiments, the second section 208 is deflated followed by deflation of the first section 206; the catheter 202 is then removed from the body lumen.

A description of some embodiments of the catheter system and method are contained in one or more of the following numbered statements:
Statement 1. A catheter system having a first configuration, a second configuration, and a third configuration, the catheter system comprising:

a catheter having a first balloon and a second balloon, the first balloon longitudinally offset from the second balloon along the length of the catheter, the first and second balloons both having expanded and unexpanded states, wherein at least one of the first balloon and the second balloon has a drug coating;

in the first configuration, the first balloon is in the unexpanded state and the second balloon is in the unexpanded state;

in the second configuration, the first balloon is in the expanded state and the second balloon is in the unexpanded state; and

in the third configuration, the first balloon is in the expanded state and the second balloon is in the expanded state.
Statement 2. The catheter system of statement 1 defining a first inflation lumen in fluid communication with the first balloon and a second inflation lumen in fluid communication with the second balloon.
Statement 3. The catheter system of statement 1 defining an inflation lumen in fluid communication with both the first and second balloons.
Statement 4. The catheter system of any one of the preceding statements, wherein the first balloon has a first inflation pressure in the expanded state and the second balloon has a second inflation pressure in the expanded state, the second inflation pressure being greater than the first inflation pressure.
Statement 5. The catheter system any one of the preceding statements, wherein the second balloon has a larger volume than the first balloon in the third configuration.
Statement 6. A catheter system having a first configuration, a second configuration, and a third configuration, the catheter system comprising:

a catheter having an inflatable balloon, the inflatable balloon having a first section and a second section, the second section longitudinally offset from the first section; the first and second sections both having expanded and unexpanded states, the second section having a drug coating, the first section being free of any drug coating;

in the first configuration, the first section is in the unexpanded state and the second section is in the unexpanded state;

in the second configuration, the first section is in the expanded state and the second section is in the unexpanded state; and

in the third configuration, the first section is in the expanded state and the second section is in the expanded state.
Statement 7. The catheter system of statement 6 defining an inflation lumen in fluid communication with both the first and second sections.
Statement 8. The catheter system of statement 6 or statement 7, wherein the first section is formed from a different material than the second section.
Statement 9. The catheter system of claim 8, wherein the material forming the second section is more stiff than the material forming the first section.
Statement 10. The catheter system of statement 8 or statement 9, wherein the material forming the second section has a greater tensile modulus than the material forming the first section.
Statement 11. The catheter system of any one of statements 6-10 wherein, in the third configuration, the first section and second section are separated by a region of reduced cross-section, the region of reduced cross-section having a smaller cross-sectional area than the first section and the second section.
Statement 12. The catheter system of any one of statements 6-12, wherein the second section has a larger volume than the first section.
Statement 13. The catheter system of any one of statements 6-12, wherein the second section is longer than the first section.
Statement 14. The catheter system of any one of statements 6-12, wherein the second section is distal to the first section.
Statement 15. The catheter system of any one of statements 6-12, wherein the first section is distal to the second section.
Statement 16. A catheter system having a first configuration, a second configuration, and a third configuration, the catheter system comprising:

a catheter having a first balloon and a second balloon, the first balloon longitudinally offset from the second balloon along the length of the catheter, the first and second balloons both having expanded and unexpanded states, wherein at least one of the first balloon and the second balloon has a drug coating;

in the first configuration, the first balloon is in the unexpanded state and the second balloon is in the unexpanded state;

in the second configuration, the first balloon is in the expanded state and the second balloon is in the unexpanded state; and

in the third configuration, the first balloon is in the expanded state and the second balloon is in the expanded state.
Statement 17. The catheter system of statement 16 defining a first inflation lumen in fluid communication with the first balloon and a second inflation lumen in fluid communication with the second balloon.
Statement 18. The catheter system of statement 16 defining an inflation lumen in fluid communication with both the first and second balloons.
Statement 19. The catheter system of statement 16, wherein the first balloon has a first inflation pressure in the expanded state and the second balloon has a second inflation pressure in the expanded state, the second inflation pressure being greater than the first inflation pressure.
Statement 20. The catheter system of statement 16, wherein the second balloon has a larger volume than the first balloon in the third configuration.
Statement 21. A catheter system having a first configuration, a second configuration, and a third configuration, the catheter system comprising:

a catheter having an inflatable balloon, the inflatable balloon having a first section and a second section, the second section longitudinally offset from the first section; the first and second sections both having expanded and unexpanded states, the second section having a drug coating, the first section being free of any drug coating;

in the first configuration, the first section is in the unexpanded state and the second section is in the unexpanded state;

in the second configuration, the first section is in the expanded state and the second section is in the unexpanded state; and

in the third configuration, the first section is in the expanded state and the second section is in the expanded state.
Statement 22. The catheter system of statement 21 defining an inflation lumen in fluid communication with both the first and second sections.
Statement 23. The catheter system of statement 21, wherein the first section is formed from a different material than the second section.
Statement 24. The catheter system of statement 23, wherein the material forming the second section is more stiff than the material forming the first section.
Statement 25. The catheter system of statement 23, wherein the material forming the second section has a greater tensile modulus than the material forming the first section.
Statement 26. The catheter system of statement 21, wherein, in the third configuration, the first section and second section are separated by a region of reduced cross-section, the region of reduced cross-section having a smaller cross-sectional area than the first section and the second section.
Statement 27. The catheter system of statement 21, wherein the second section has a larger volume than the first section.
Statement 28. The catheter system of statement 21, wherein the second section is longer than the first section.
Statement 29. The catheter system of statement 21, wherein the second section is distal to the first section.
Statement 30. The catheter system of statement 21, wherein the first section is distal to the second section.
Statement 31. A method of deploying a catheter system within a bodily lumen having a lumen wall, the method comprising:

providing a catheter having a first balloon and a second balloon, the first balloon longitudinally offset from the second balloon, the second balloon has thereon a drug coating comprising a drug, the first balloon being free of any drug coating;

deploying the first balloon within the bodily lumen upstream of the second balloon, wherein deployment of the first balloon stagnates the fluid within the bodily lumen downstream of the first balloon;

deploying the second balloon within the bodily lumen in the stagnated fluid; and

emitting the drug;

transferring the drug to the lumen wall;

deflating the first and second balloons; and

removing the catheter from the bodily lumen.
Statement 32. The method of statement 31, wherein the catheter system includes a first inflation lumen and the step of deploying the first balloon within the bodily lumen includes conveying an inflation fluid through the first inflation lumen into the first balloon.
Statement 33. The method of statement 32, wherein the catheter system includes a second inflation lumen and the step of deploying the second balloon within the bodily lumen includes conveying an inflation fluid through the second inflation lumen into the second balloon.
Statement 34. The method of statement 31, wherein the step of deploying the first balloon within the bodily lumen includes conveying a saline solution into the first balloon.
Statement 35. The method of statement 32, wherein the step of deploying the second balloon within the bodily lumen includes conveying a saline solution into the second balloon.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A catheter system having a first configuration, a second configuration, and a third configuration, the catheter system comprising:
a catheter having a first balloon and a second balloon, the first balloon longitudinally offset from the second balloon along the length of the catheter, the first and second balloons both having expanded and unexpanded states, wherein at least one of the first balloon and the second balloon has a drug coating;
in the first configuration, the first balloon is in the unexpanded state and the second balloon is in the unexpanded state;
in the second configuration, the first balloon is in the expanded state and the second balloon is in the unexpanded state; and
in the third configuration, the first balloon is in the expanded state and the second balloon is in the expanded state.

2. The catheter system of claim 1 defining a first inflation lumen in fluid communication with the first balloon and a second inflation lumen in fluid communication with the second balloon.

3. The catheter system of claim 1 defining an inflation lumen in fluid communication with both the first and second balloons.

4. The catheter system of claim 1, wherein the first balloon has a first inflation pressure in the expanded state and the second balloon has a second inflation pressure in the expanded state, the second inflation pressure being greater than the first inflation pressure.

5. The catheter system of claim 1, wherein the second balloon has a larger volume than the first balloon in the third configuration.

6. A catheter system having a first configuration, a second configuration, and a third configuration, the catheter system comprising:
a catheter having an inflatable balloon, the inflatable balloon having a first section and a second section, the second section longitudinally offset from the first section; the first and second sections both having expanded and unexpanded states, the second section having a drug coating, the first section being free of any drug coating;
in the first configuration, the first section is in the unexpanded state and the second section is in the unexpanded state;
in the second configuration, the first section is in the expanded state and the second section is in the unexpanded state; and
in the third configuration, the first section is in the expanded state and the second section is in the expanded state.

7. The catheter system of claim 6 defining an inflation lumen in fluid communication with both the first and second sections.

8. The catheter system of claim 6, wherein the first section is formed from a different material than the second section.

9. The catheter system of claim 8, wherein the material forming the second section is more stiff than the material forming the first section.

10. The catheter system of claim 8, wherein the material forming the second section has a greater tensile modulus than the material forming the first section.

11. The catheter system of claim 6, wherein, in the third configuration, the first section and second section are separated by a region of reduced cross-section, the region of reduced cross-section having a smaller cross-sectional area than the first section and the second section.

12. The catheter system of claim 6, wherein the second section has a larger volume than the first section.

13. The catheter system of claim 6, wherein the second section is longer than the first section.

14. The catheter system of claim 6, wherein the second section is distal to the first section.

15. The catheter system of claim 6, wherein the first section is distal to the second section.

16. A method of deploying a catheter system within a bodily lumen having a lumen wall, the method comprising:
providing a catheter having a first balloon and a second balloon, the first balloon longitudinally offset from the second balloon, the second balloon has thereon a drug coating comprising a drug, the first balloon being free of any drug coating;
deploying the first balloon within the bodily lumen upstream of the second balloon, wherein deployment of the first balloon stagnates the fluid within the bodily lumen downstream of the first balloon;
deploying the second balloon within the bodily lumen in the stagnated fluid; and
emitting the drug;
transferring the drug to the lumen wall;
deflating the first and second balloons; and
removing the catheter from the bodily lumen.

17. The method of claim 16, wherein the catheter system includes a first inflation lumen and the step of deploying the first balloon within the bodily lumen includes conveying an inflation fluid through the first inflation lumen into the first balloon.

18. The method of claim 17, wherein the catheter system includes a second inflation lumen and the step of deploying the second balloon within the bodily lumen includes conveying an inflation fluid through the second inflation lumen into the second balloon.

19. The method of claim 16, wherein the step of deploying the first balloon within the bodily lumen includes conveying a saline solution into the first balloon.

20. The method of claim 17, wherein the step of deploying the second balloon within the bodily lumen includes conveying a saline solution into the second balloon.
